(19) United States
(12) Patent Application Publication
(10) Pub. No.: US 2010/0286546 A1
(43) Pub. Date: Nov. 11, 2010

(54) GARMENT FOR DETECTING RESPIRATORY MOVEMENT

(75) Inventors: Andreas Tobola, Hemhofen (DE); Christian Weigand, Fuerth (DE)

Correspondence Address: SCHOPPE, ZIMMERMANN , STOCKELER & ZINKLER;C/O KEATING & BENNETT, LLP, 1800 Alexander Bell Drive, SUITE 200, Reston, VA, 20191, US

(73) Assignee: Fraunhofer-Gesellschaft zur Foerderung der angewandten Forschung e.V., Munich (DE)

(21) Appl. No.: 12/742,260

(22) PCT Filed: Nov. 4, 2008
(86) PCT No.: PCT/EP2008/009286

§ 371 Date: Jun. 17, 2010

(30) Foreign Application Priority Data
Nov. 12, 2007 (DE) 10 2007 053 843.1

(51) Int. Cl. A61B 5/08

(52) U.S. Cl. 600/534

(57) ABSTRACT

A garment for detecting respiratory movement of a living being, wherein the garment can be pulled over the thorax of the living being, including an electric conductor integrable at the height of the thorax of the living being into the garment, which is attached to the garment in order to change an electrically measurable characteristic in dependence on the respiratory movement of the thorax, and a holder for fixing evaluation electronics integrable into the garment, which can be coupled to the electric conductor and which is implemented to detect the electrically measurable characteristic and which further includes an interface for outputting or storing data derived from the electrically measurable characteristic.

### BACKGROUND OF THE INVENTION

The present invention relates to devices for monitoring changes of a state of motion of parts of a living being and, in particular, devices for detecting respiratory movements of a living being.

The variation of the girth at least one part of the torso of a living being is referred to as breathing effort. Typically, the breathing effort is measured at two positions of the torso, abdomen and thorax, and mapped as a signal and stored. The breathing effort maps the tension or relaxation, respectively, of the lung muscles. A change in breathing effort—which means tension and relaxation, respectively, of the lung muscles—can also take place when actually no breathing flow, which means no exchange of breathing gases, exists. Normally, however, the breathing effort is connected to the breathing flow.

For example, a graphical mapping of the signals of the breathing effort allows doctors to directly interpret a vital state of a living being regarding the lung function. Additionally, further processing of this signal to vital parameters is possible. This can, for example, be breathing frequency, breathing volume, breathing depth or the general course of the breathing. Detecting the breathing effort can be of use in somnology (sleep research), sports medicine or home monitoring. One example for home monitoring usage is the phenomena of sudden infant death, called SIDS (Sudden Infant Death Syndrome) in medical literature. Reason for this sudden infant death is a central respiratory standstill of unknown origin. This example from the field of medicine is particularly relevant in that SIDS threatens all children in the first year of their lives. Sudden infant death is responsible for almost half of all causes of death between the second and the twelfth month of life.

Known methods for detecting the breathing effort are based, for example, on sensors or measurement value sensors, respectively, recording respiratory movements, devices for measuring the thorax impedance, sensors for heart activity, such as EKG devices, induction plethysmographs or pulse oximeters. In several of these methods, the breathing effort is measured by means of bands or belts, respectively, which are applied around one part of the body. The methods for detecting the breathing effort can be divided into two classes, stress-free methods and methods necessitating a certain tension of the belts. One example of a stress-free method is induction plethysmography, where the belts are applied loosely around the selected parts of the body. In contrary to this, in plethysmography of breathing by means of strain measurement strips (strain gauge plethysmography), much more tension of the belt is necessitated, which can be unpleasant for a patient. Independent of the measurement method, such belts can be worn under the clothing, but have to be connected to a signal processing electronics via cables. A negative characteristic of the belts is, for example, that they slip during wearing. After the application, the bands are positioned at a certain position of the body, they can, however, change their position during the course of the measurement, for example, due to movement or unevenness of the respective part of the body.

### SUMMARY

According to an embodiment, a garment for detecting respiratory movement of a living being, wherein the garment can be pulled over the thorax of the living being, may have: a straight electric conductor, which is integrable into the garment at the height of the thorax of the living being, which is attached to the garment in order to change an inductance of the electric conductor in dependence on the respiratory movement of the thorax, wherein the electric conductor is an elastic electrically conductive fiber, which can expand in dependence on the respiratory movement and thereby experiences a change of the inductance when the garment is pulled over the thorax of the living being; and a holder for fixing evaluation electronics, which is integrable into the garment; and the integrable evaluation electronics, which can be coupled to the elastic electrically conductive fiber and is implemented to detect a change in inductance of the elastic electrically conductive fiber and further has an interface for outputting or storing the data derived from the inductance.

Another embodiment may have a usage of the inventive garment for detecting respiratory movement of a living being, wherein the integrable evaluation electronics is fixed to the garment by means of the holder, wherein the integrable evaluation electronics is coupled to the elastic electrically conductive fiber for detecting the change in inductance of the elastic electrically conductive fiber.

According to another embodiment, a method for producing a garment for detecting respiratory movement of a living being, wherein the garment can be pulled over the thorax of the living being, may have the steps of: integrating an elastic electrically conductive fiber into the garment, such that the elastic electrically conductive fiber can expand in dependence on the respiratory movement of the thorax when the garment is pulled over the thorax of the living being and thereby experiences change of an inductance of the elastic electrically conductive fiber; and attaching a holder to the garment for fixing evaluation electronics integrable into the garment; and providing the integrable evaluation electronics, which can be coupled to the elastic electrically conductive fiber and which is implemented to detect a change in inductance of the elastic electrically conductive fiber and which further has an interface for outputting or storing data derived from the inductance.

It is the finding of the present invention that slipping of sensors for measuring breathing effort integrated in belts or bands can be avoided by integrating such sensors, such as inductively operating sensors into a garment, which can be pulled over the torso and, in particularly, over the thorax of the living being. Thereby, in embodiments of the present invention, the sensor is an electric conductor structure, which is integrated into the garment such that the sensor is at the same height as the thorax when the garment is put on. If the garment, such as a T-shirt or a playsuit, is put-on and relatively tight at the body of a person, slipping of the sensor in the form of the electric conductor can be largely prevented.

According to an embodiment of the present invention, the garment has a holder for evaluation electronics that can be coupled to the sensor and is integrable into the garment. This holder can, for example, be some type of pocket, into which the evaluation electronics, for example in the form of an integrated circuit, can be inserted. The evaluation electronics is implemented to output or store data derived from the electrically measurable characteristic.

The breathing effort of a living being changes the diameter at different positions of the torso and, hence, also the diameter of the sensor guided around the torso in the form of an electric conductor integrated in the garment. Due to this change of the diameter or the perimeter, respectively, of the electric conductor, an electrically measurable characteristic of the electric conductor is changed in dependence on the breathing effort of the thorax. If the evaluation electronics is coupled to the electric conductor, the same can detect the electrically measurable characteristic and output or store data derived therefrom via an interface integrated in the evaluation electronics. The interface can be a wire interface or a wireless interface for radio transmission.

According to embodiments, breathing effort changes the inductance of the electric conductor, which, when the garment is put on, substantially forms a conductor loop around the torso of the living being, wherein the inductance of the electric conductor depends on the diameter or perimeter of the same, respectively.

In other embodiments of the present invention, the breathing effort changes the electric resistance of the electric conductor. Thereby, the electric conductor comprises at least one elastic fiber changing its electric resistance or inductance with expansion and contraction.

Hence, embodiments of the present invention relate to an independent garment by which the breathing effort and/or vital parameters derivable therefrom can be detected and determined. Further, these vital parameters can be wirelessly transmitted or optionally locally stored in real time by means of the evaluation electronics. The breathing effort can be measured at least on one or also on several parts of the body. In the case of several parts of the body, the garment according to an embodiment has a further electric conductor at the height of the abdomen (when the garment is put on) of the living being, which can be coupled to the evaluation electronics.

The garment is equally suitable for monitoring newborn babies, infants and adults. Additionally, it is comfortable and contamination-free. The evaluation electronics and/or its energy supply can be taken out of the garment for washing. Due to the sensors integrated into the garment, exact positioning of the sensors is possible. Additionally, the sensors do no longer slip during measurement. This has the great advantage that the breathing amplitude can be stated with one unit, for example, change of the perimeter of a part of a body in centimeter or change of the volume of the part of the body in cm³.

By the exact positioning of the electric conductors as sensors and by fixing these sensors at a fixed position, more exact and reliable measurement of the respiratory movement becomes possible. The measurement becomes more reliable due to the fixed position by preventing slipping of the electric conductors into a region of the body where physiologically no breathing can be detected. By eliminating the influences resulting from slipping of the electric conductors, the measured quantity can, above that, be stated with one unit.

A further advantage is the securing of the measurement arrangement at the patient. The garment simply needs to be pulled on. In contrary to this, conventional systems, consisting of their individual parts, have to be positioned, wired and fixed to the clothing of the patient.

Due to the exact positioning of the sensors as well the resulting exact measurement of the change of the perimeter, quantities, such as breathing frequency, breathing depth and, in particular, breathing volume (with previous calibration) can be determined very accurately.

When using elastic electric conductors or fibers, respectively, which are woven into the garment in a straight manner, i.e. in a circular or ring-shape around the thorax, additionally, wearing comfort and/or esthetics can be improved. Such a fiber can be integrated into the garment in a very discreet manner and takes up little space, whereby the freedom of movement of a wearer of the garment is hardly affected. By discreetly incorporating one or several elastic fibers, the garment can no longer be differentiated from a “normal” tight garment, whereby an onlooker cannot recognize the functionality of the garment, such as monitoring vital parameters. Thus, possibly, inhibitions with regard to wearing such a garment in public might be overcome.

Other elements, features, steps, characteristics and advantages of the present invention will become more apparent from the following detailed description of the preferred embodiments with reference to the attached drawings.

### BRIEF DESCRIPTION OF THE DRAWINGS

Embodiments of the present invention will be detailed subsequently referring to the appended drawings, in which:

FIG. 1 is a schematic illustration of a garment for detecting respiratory movement according to an embodiment of the present invention;

FIG. 2 is an illustration of a conductor loop carrying magnetic flow;

FIG. 3 is an illustration of a breathing curve plotted over the time;

FIG. 4 is a schematic illustration of a garment for detecting respiratory movement according to a further embodiment of the present invention;

FIG. 5 is a schematic illustration of a garment for detecting respiratory movement having elastic electric conductors according to a further embodiment of the present invention;

FIG. 6 is an image of evaluation electronics according to an embodiment of the present invention;

FIG. 7 is an image of a person wearing a garment according to an embodiment of the present invention; and

FIG. 8 is an image of a playsuit for babies according to an embodiment of the present invention.

### DETAILED DESCRIPTION OF THE INVENTION

Regarding the following description, it should be noted that in the different embodiments, equal or similar functional elements have the same reference numerals and, hence, descriptions of these functional elements are interchangeable in the different embodiments illustrated below.

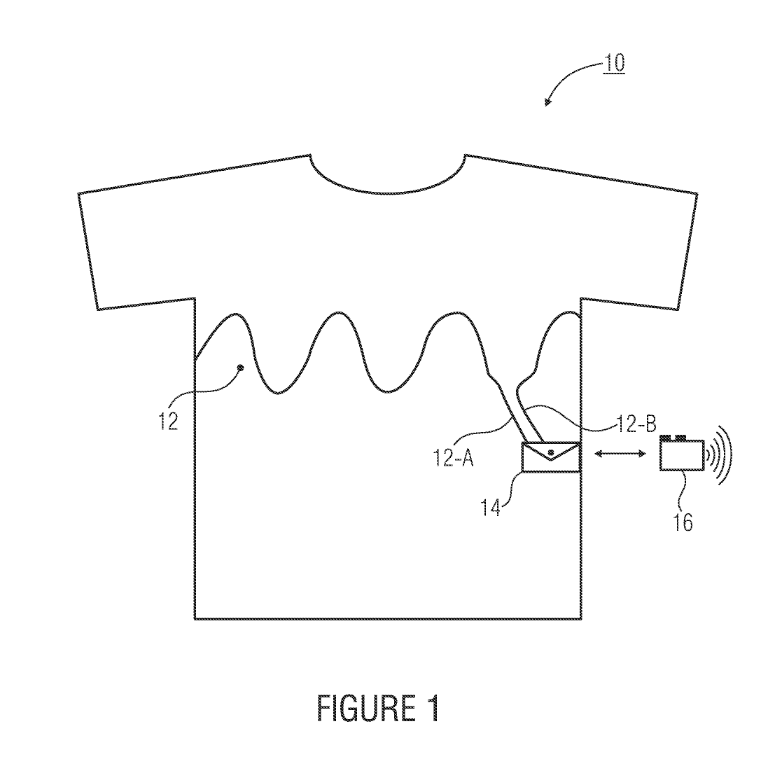

FIG. 1 shows a schematic illustration of a garment 10 for detecting respiratory movement of a living being.

Although in the following the detection of the respiratory movement of human beings is mentioned, embodiments of the present invention are, in no way, limited to humans, but can also be correspondingly applied to animals, in particular, mammals.

The garment 10 is implemented according to embodiments of the present invention in order to be pulled over the thorax of a person or living being, respectively. In embodiments of the present invention, the garment 10 can not only be pulled over the thorax, but over the complete torso of a person. The garment can, for example, be implemented as a tight T-shirt or, for infants, as a tight playsuit. It is important that the garment is as tight as possible at the torso of the person. This can be obtained, for example, by the fact that the garment 10 consists of fabric that is as elastic as possible, which can expand and contract with the respiratory movement. Such elastic fabrics are available in a large variety.

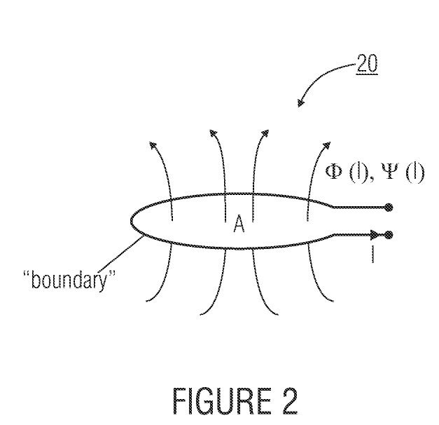

If the garment 10 is assumed to be pulled over the torso of the person, then the garment 10 includes, at the height of the thorax of the person, a sensor in the form of an electric conductor 12 integrated in the garment 10, which is attached to the garment 10 such that in dependence on the respiratory movement of the thorax, an electrically measurable characteristic of the electric conductor 12 can change. Thereby, the electric conductor 12 is integrated into the garment 10 such that the electric conductor 12 reaches from a first line end 12-A, when the garment is pulled-on, around the thorax of the person to a second line end 12-B, similar to a conductor loop as schematically shown in FIG. 2.

The two line ends 12-A, 12-B are guided to a holder 14 integrated into the garment. The holder 14 serves to fix evaluation electronics 16 integrable into the garment, which can be coupled to the electric conductor 12 via the two line ends 12-A, 12-B and which is implemented to detect the electrically measurable characteristic of the electric conductor 12. Further, the evaluation electronics comprises an interface for outputting or storing data derived from the electrically measurable characteristic. The interface of the evaluation electronics 16 can be a wireless interface for radio transmission, as schematically illustrated in FIG. 1. Also, obviously, a wired interface or a storage interface is possible.

According to embodiments, the holder 14 can be some type of pocket into which the evaluation electronics can be inserted and coupled to the two line ends 12-A, 12-B. Coupling the line ends 12-A, 12-B with the evaluation electronics 16 can be performed via a plug connection or, for example, via push buttons. For fixing the evaluation electronics 16 in the pocket 14, according to an embodiment of the present invention, the pocket 14 is provided with a closing mechanism, such as a Velcro fastener or a push button. The evaluation electronics and/or its energy supply can be taken out of the pocket for washing.

Alternatively, the evaluation electronics 16 could also be implemented in a watertight manner, for example by casting the evaluation electronics 16 into epoxy resin, and could be firmly integrated or sewn-in into the garment 10. In this case, the holder 14 has to be provided for a source of energy, such as a battery or an accumulator for the evaluation electronics 16 at the garment 10.

According to an embodiment of the present invention, the electric conductor 12 is a wire, which can be insulated and which is sewn into the garment 10. Thereby, the insulated wire 12 can be sewn into the garment 10 in different ways. The wire 12 can, for example, run directly through the fabrics of the garment 10 or run on the fabric in a specifically provided lug. In order to be able to follow the respiratory movement of the thorax of the person, i.e. in order to be able to expand and contract, the electric conductor 12 is integrated into the garment 10, for example, in a sinusoidal shape, meander shape or a zigzag shape.

Due to the fact that the electric conductor 12 runs around the thorax of the person when the garment is put on, the same forms a conductor loop schematically shown in FIG. 2.

When a signal, for example, in the form of current or voltage, is applied to the two line ends 12-A, 12-B of the conductor loop 12, a magnetic field results and, hence, a magnetic flow. The ratio of magnetic flow through the conductor loop to the current in the conductor loop is called inductance L. Assumed that the diameter d of the used electric conductor 12 is very small in comparison with the diameter D of the conductor loop formed by the electric conductor 12 (d/D<0.001), i.e. the diameter of the thorax, a relatively simple approximate solution can be used for the inductance of the conductor loop. Accordingly, the inductance L results in

L=μ₀·R·ln(2R/d).

Thereby, R=D/2 is the radius of the conductor loop, d the diameter of the used electric conductor 12 and μ₀ is the magnetic field constant.

By the respiratory movement of the thorax, the radius or perimeter, respectively, of the conductor loop 12 tightly applied to the thorax changes and, hence, its inductance L. The inductance L of the electric conductor 12 alterable with the respiratory movement forms, according to an embodiment of the present invention, the inductance of a LC parallel resonant circuit. According to an embodiment of the present invention, the LC parallel resonant circuit is part of an electric circuit known under the name of Colpitts circuit or Colpitts oscillator, respectively. Since merely the inductance L of the electric conductor 12 varies, the same substantially determines the frequency generated by the Colpitts oscillator. Thereby, apart from the electric conductor 12, the Colpitts oscillator resides within the evaluation electronics 16. Thus, the respiratory movement of the person can be converted to a frequency of the oscillator circuit depending on the respiratory movement of the person by means of the variable inductance L of the electric conductor 12.

According to embodiments of the present invention, the evaluation electronics 16 comprises a frequency counter for detecting the frequency of the oscillator circuit varying due to the breathing effort. If the thorax of the living being expands during inhalation, the inductance L of the electric conductor 12 increases due to the increasing radius R of the conductor loop around the thorax formed by the electric conductor 12. Thereby, the frequency generated by the oscillator circuit with the LC parallel resonant circuit is reduced. This applies vice-versa for the exhalation.

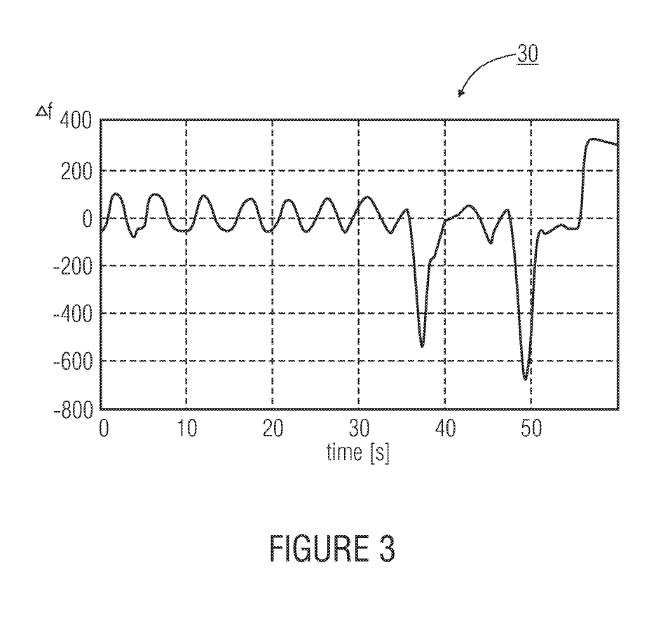

By monitoring the frequency of the Colpitts oscillator, a breathing curve depending on the respiratory movement of the person can be illustrated. Such a breathing curve is exemplarily shown in FIG. 3.

In the breathing curve shown in FIG. 3, a thorax perimeter deviation ΔU from a nominal thorax perimeter is plotted against the time. It can be seen that up to a time period of approximately 35 seconds, a relatively normal, even breathing motion prevails. At the time of approximately 35 seconds, however, a drop of the breathing curve can be seen, which is due to strong exhalation. In a time period between 40 seconds and approximately 47 seconds, the breathing curve 30 is normalized again in order to experience a renewed drop at approximately 50 seconds.

It is obvious that with the illustrated concept, a relatively exact and reliable monitoring of the breathing activity of a living being can be obtained

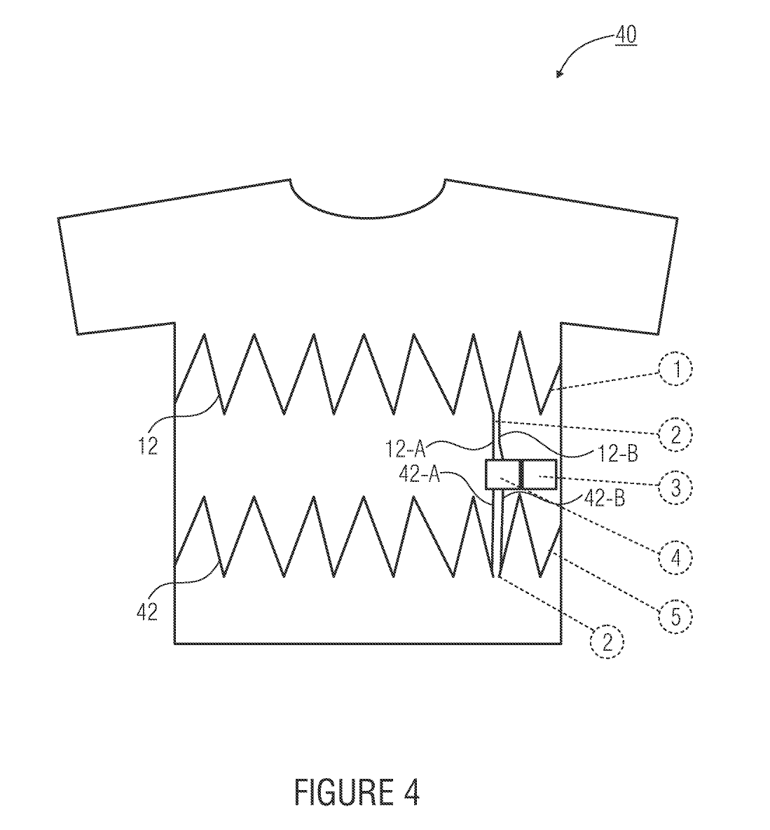

FIG. 4 shows a schematic illustration of a further embodiment of a garment for detecting respiratory movement of a person.

For detecting the breathing activity even more reliably, the garment 40 comprises, compared with the garment 10 shown in FIG. 1, a further electric conductor 42 in addition to the electric conductor 12, which is integrated into the garment 40 in order to change an electrically measurable characteristic in dependence on the breathing motion of the abdomen. According to embodiments, the electrically measurable characteristic of the further conductor 42 is also its inductance. The further conductor 42 can also be coupled to the evaluation electronics 16 via its line ends 42-A, 42-B. According to embodiments, the further conductor 42 is integrated into the garment 40 in the same manner as the first electric conductor 12.

According to embodiments, the further electric conductor 42 can be coupled to a further Colpitts oscillator circuit via its line ends 42-A, 42-B for obtaining a further frequency response in dependence on the respiratory movement of the living being. Further, the frequency response can be converted into other vital parameters, such as a perimeter of the part of the body, etc. The further electric conductor 42 can increase the reliability of the measurement method further.

The two electric conductors 12, 42 are woven-in or sewn-in as lace around the whole torso in a predetermined height, for example, as individual conductors. If they are normal, non-flexible electric lines, such as metallic wires, a zigzag-shaped, sinusoidal-shaped or meander-shaped arrangement of the lines is advantageous in order to allow for the respiratory movement. The electric lines 12, 42 can each be interrupted at any position. This interruption results in the respective line ends and serves as a terminal for the evaluation electronics 16.

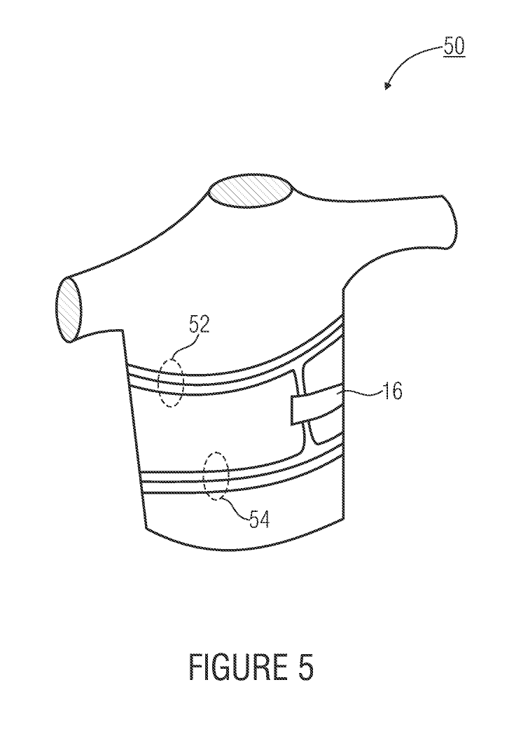

According to a further embodiment of the present invention, elastic fibers can also be used as conductors 12 and/or 42, which, for example, change their electric resistance R or their inductance L during expansion. When using such elastic fibers, same do not have to be integrated into the garment in a zigzag shape, sinusoidal shape or meander shape, but can be woven in straight, i.e. in a circular or ring shape around the thorax as schematically shown in FIG. 5. Thereby, via the number of adjacently running elastic electric conductors 52, 54 or the number of windings of the elastic electric conductors 52, 54, a nominal resistance can be adjusted, which is adapted to the evaluation electronics 16. By the respiratory movement of the thorax or abdomen, respectively, the corresponding electric resistances of the expandable electric conductors 52, 54 change, such that in this case, for example, an above-described LC parallel resonant circuit can be attenuated in a variable manner. A change of resistance can be detected and mapped to a signal similar to the one shown in FIG. 3 in many ways known to the person skilled in the art.

By using an elastic electric conductor, which is woven into the garment in a straight manner, i.e. in a circular or ring shape around the thorax, the wearing comfort and esthetics can be significantly improved. According to the invention, an electrically conductive elastic fiber is used as electric conductor. Such a fiber can be integrated significantly less noticeably into the garment than a wire running in a curve-shape. Such a fiber takes up less space, which interferes less with the freedom of movement of the wearer of the garment. This means the wearing comfort is increased. By discreetly inserting one or several elastic fibers into the garment, the garment cannot be differentiated from a “normal” tight garment, which makes it impossible for an onlooker to detect the functionality of the garment, i.e. measuring vital parameters. This might also reduce the inhibitions of a patient to wear such a garment in public.

The evaluation electronics 16 serves for detecting and evaluating the measurement values of sensors 12, 42, processing the detected signals and providing data via a wireless or wired interface. Alternatively, the detected data can also be stored. A source of energy, for example an accumulator, supplies the evaluation electronics 16 with energy. The garment 10, 40 should be adjusted to the living being or the test person, respectively, and should be tight, such as a vest or a sports shirt.

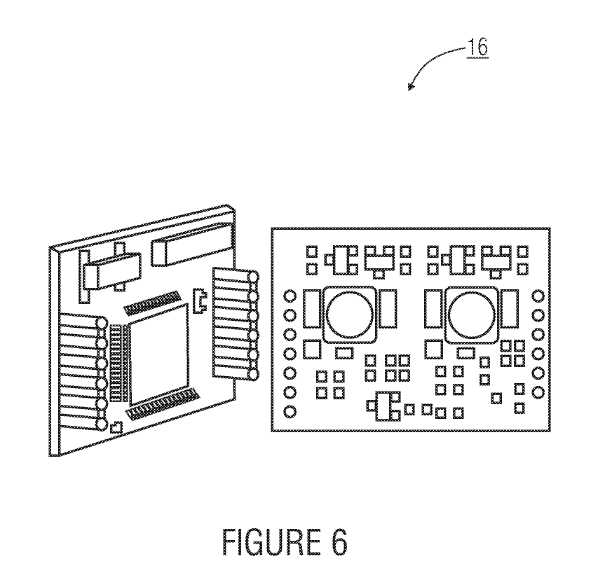

Evaluation electronics 16 according to an embodiment of the present invention is illustrated in FIG. 6.

The evaluation electronics 16 is housed on a small and light circuit board. According to an embodiment, the circuit board has dimensions of approximately 2×2 cm and can, hence, be easily integrated into the garment. As has already been described above, the evaluation electronics can, for example, comprise a Colpitts circuit, wherein the inductance of the LC parallel resonant circuit is formed by the electric conductor. In this case, the evaluation electronics 16 has a frequency counter for detecting the frequency generated by the Colpitts oscillator.

Instead of detecting an inductance of the electric conductor changing in dependence on the respiratory movement, according to embodiments of the present invention, the evaluation electronics 16 can also be formed to detect, for example, a change of the electric resistance R of an electric conductor. This can, for example, be provided when elastic or expandable fibers are used as electric conductors, which change their electric resistance R in dependence on their expansion.

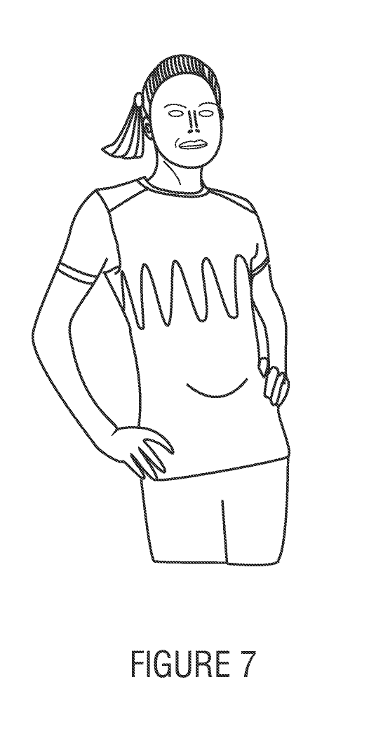

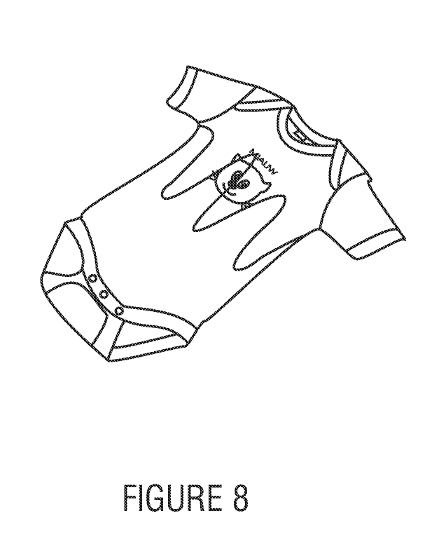

Two garments according to embodiments of the present invention are illustrated exemplarily in FIGS. 7 and 8.

FIG. 7 shows an inventive sports shirt with integrated electric conductors as measurement value sensors and integrated evaluation electronics. Here, it should be noted that the sports shirt is applied relatively tightly at the torso of the wearer.

FIG. 8 shows a baby playsuit with integrated electric conductors as measurement value sensor. As has been already mentioned above, by using such a baby playsuit with integrated breathing detection, sudden infant death can be detected and prevented.

The inventive concept is substantially based on detecting a change of the perimeter of a woven-in or sewn-in electric conductor. This change of the perimeter affects a change of the inductance L or the resistance R of the electric conductor. The inductance L or the resistance R can be continuously detected and digitalized by the evaluation electronics.

With an overall system comprising a garment with an integrated electric conductor and attached evaluation electronics, continuous measurement of the body perimeter at several fixed positions, but at least one, is possible. By fixing the electric conductors in the garment, the measurement locations do not change during usage. Thereby, exact measurement of the breathing effort and the vital parameters derivable therefrom becomes possible.

According to embodiments of the present invention, an evaluation electronics in the form of a small printed circuit board is used for evaluating the detected signals, which can also be integrated into the garment. According to embodiments, the circuit board has a radio interface for transmitting the measurement values by radio to a display, alarm and/or recording unit. This can, for example, be a wristwatch, a PDA (Personal Digital Assistant) or a PC (Personal Computer). In medical applications, an alarm can be triggered when the breathing stops. In sports, for example, the breathing frequency can be detected by the inventive concept and all data can be recorded for later analysis.

In summary, it should be noted that the present invention is not limited to the respective individual parts of the garment or the discussed procedure, since these individual parts and methods can vary. The terms used are merely intended for describing specific embodiments and are not used in a limiting sense. When singular or indefinite articles are used in the description and in the claims, they also relate to the plurality of these elements as long as the overall context does not specify anything else. The same applies vice-versa.

While this invention has been described in terms of several advantageous embodiments, there are alterations, permutations, and equivalents which fall within the scope of this invention. It should also be noted that there are many alternative ways of implementing the methods and compositions of the present invention. It is therefore intended that the following appended claims be interpreted as including all such alterations, permutations, and equivalents as fall within the true spirit and scope of the present invention.

1-13. (canceled)

14. Garment for detecting respiratory movement of a living being, wherein the garment can be pulled over the thorax of the living being, comprising:
a straight electric conductor, which is integrable into the garment at the height of the thorax of the living being, which is attached to the garment in order to change an inductance of the electric conductor in dependence on the respiratory movement of the thorax, wherein the electric conductor is an elastic electrically conductive fiber, which can expand in dependence on the respiratory movement and thereby experiences a change of the inductance when the garment is pulled over the thorax of the living being; and
a holder for fixing evaluation electronics, which is integrable into the garment; and
the integrable evaluation electronics, which can be coupled to the elastic electrically conductive fiber and is implemented to detect a change in inductance of the elastic electrically conductive fiber and further comprises an interface for outputting or storing the data derived from the inductance.

15. Garment according to claim 14, wherein the integrable evaluation electronics is implemented such that the inductance of the elastic electrically conductive fiber variable with the respiratory movement forms an inductance of an LC parallel resonant circuit, which is part of a Colpitts oscillator circuit, wherein the Colpitts oscillator circuit is within the integrable evaluation electronics.

16. Garment according to claim 15, wherein the integrable evaluation electronics comprises a frequency counter for detecting a frequency of the Colpitts oscillator circuit varying due to the respiratory movement.

17. Garment according to claim 14, wherein the electric conductor is integrated into the garment such that an area surrounded by the elastic electrically conductive fiber is dependent on the respiratory movement when the garment is pulled over the thorax of the living being.

18. Garment according to claim 14, wherein the evaluation electronics is fixed in the holder such that the evaluation electronics is part of the garment, wherein the evaluation electronics is implemented in a watertight manner.

19. Garment according to claim 14, wherein the integrable evaluation electronics comprises a wireless interface for radio communication.

20. Garment according to claim 14, wherein a fabric of the garment is elastic for allowing tight application of the electric conductor to the torso of the living being.

21. Garment according to claim 14, wherein the garment can additionally be pulled over the abdomen of the living being and comprises a further electric conductor at the height of the abdomen of the living being, which is attached to the garment in order to change an electrically measurable characteristic in dependence on the respiratory movement of the abdomen and wherein the further electric conductor can be coupled to the integrable evaluation electronics.

22. Usage of a garment for detecting respiratory movement of a living being, wherein the garment can be pulled over the thorax of the living being, the garment comprising:
a straight electric conductor, which is integrable into the garment at the height of the thorax of the living being, which is attached to the garment in order to change an inductance of the electric conductor in dependence on the respiratory movement of the thorax, wherein the electric conductor is an elastic electrically conductive fiber, which can expand in dependence on the respiratory movement and thereby experiences a change of the inductance when the garment is pulled over the thorax of the living being; and
a holder for fixing evaluation electronics, which is integrable into the garment; and
the integrable evaluation electronics, which can be coupled to the elastic electrically conductive fiber and is implemented to detect a change in inductance of the elastic electrically conductive fiber and further comprises an interface for outputting or storing the data derived from the inductance,
wherein the integrable evaluation electronics is fixed to the garment by means of the holder, wherein the integrable evaluation electronics is coupled to the elastic electrically conductive fiber for detecting the change in inductance of the elastic electrically conductive fiber.

23. Method for producing a garment for detecting respiratory movement of a living being, wherein the garment can be pulled over the thorax of the living being, comprising:
integrating an elastic electrically conductive fiber into the garment, such that the elastic electrically conductive fiber can expand in dependence on the respiratory movement of the thorax when the garment is pulled over the thorax of the living being and thereby experiences change of an inductance of the elastic electrically conductive fiber; and
attaching a holder to the garment for fixing evaluation electronics integrable into the garment; and
providing the integrable evaluation electronics, which can be coupled to the elastic electrically conductive fiber and which is implemented to detect a change in inductance of the elastic electrically conductive fiber and which further comprises an interface for outputting or storing data derived from the inductance.
